(19) United States
(12) Patent Application Publication
(10) Pub. No.: US 2011/0223588 A1
(43) Pub. Date: Sep. 15, 2011

(54) Solid Phase Nucleic Acid Extraction From Small Sample Volumes, and Release of Controlled Quantities

(75) Inventor: Chiu W. Chau, Edison, NJ (US)

(73) Assignee: BioSample LLC, Edison, NJ (US)

(21) Appl. No.: 13/017,213

(22) Filed: Jan. 31, 2011

Related U.S. Application Data
(60) Provisional application No. 61/311,825, filed on Mar. 9, 2010.
(60) Provisional application No. 61/407,197, filed on Oct. 27, 2010.
(60) Provisional application No. 61/410,045, filed on Nov. 4, 2010.

(51) Int. Cl. C12Q 1/68

(52) U.S. Cl. 435/6.1

(57) ABSTRACT

Products for and a method of capturing and storing nucleic acid from patient blood, plants or other samples (e.g., purified DNA or RNA), for use in analysis of nucleic acid are described. The products are made by heating a mixture of magnetic beads, silica particles, alkyl silicate (e.g., Silbond 4, Silbond Corp., Weston Mich.) and polyethylene resin particles. The quantity of nucleic acid adsorbed by the product is controlled by the surface area of silica available for binding to nucleic acid, which in turn is controlled by: the overall volume of the product, the ratio of the volume of polyethylene resin particles to the volumes of silica particles and alkyl silicate; and the sizes of silica particles (smaller particles have a larger surface area per unit volume). Controlling and limiting of the amount of nucleic acid captured by the product avoids the disadvantages associated with excess DNA/RNA for PCR amplification.

### RELATED APPLICATIONS

This application claims priority to U.S. Provisional Nos. 61/311,825, filed Mar. 9, 2010; 61/407,197, filed Oct. 27, 2010; and 61/410,045, filed Nov. 4, 2010.

### FIELD OF THE INVENTION

The invention relates to solid phase capturing and storing of DNA or RNA, from a small sample volume, and releasing a controlled quantity of DNA or RNA from the solid phase.

### BACKGROUND

Known extraction methods of nucleic acid from patient samples, e.g., blood and body fluids or feces or tissues, or from plant products, including leaves, include adsorption onto adherent surfaces, like filter paper or silica-coated beads. The nucleic acid is then eluted from the adherent surface and subject to amplification and analysis. Additives, including chaotropic agents to fight contamination, may be included as elution ingredients. These methods cannot extract from microliter volumes of solution, or from limited numbers of cells, as the processing of the extracted samples is done at macro scale. Also, elution of nucleic acids is preferably avoided as it is an additional step, slowing or increasing the cost of the analysis process, making the process more difficult to automate, and it is a step where contaminants can readily be introduced.

Advances in amplification methods have changed the quantities of DNA/RNA required for amplification. In the past, a relatively large quantity—in the order of 1 g of DNA—was needed to perform PCR amplification, and DNA analysis. Today, as little as 5 ng, or a concentration of about 2 ng/μl, is typically required, and if those quantities are exceeded, the amplification efficiency drops and in many case, the amplification reaction cannot be completed. Therefore, it is desirable to limit the DNA and RNA captured and/or to limit the quantities eluted, so as to prevent having excess nucleic acid overwhelm the amplification reaction.

### SUMMARY

Products for and a method of capturing nucleic acid from patient blood, plants or other samples (e.g., purified DNA or RNA), for use in analysis of nucleic acid are described. The products are made by heating a mixture of magnetic beads, silica particles, alkyl silicate (e.g., Silbond 4, Silbond Corp., Weston Mich.) and polyethylene resin particles so as to weld the polyethylene resin particles together with silica particles and alkyl silicate, with the magnetic beads embedded in the melt, such that, as a result of the melting/welding of the polyethylene resin particles, fluid pathways are formed between the welded particles. If the product is immersed in a lysed solution of patient blood or another type of sample, or if blood or fluid is flowed through the product, nucleic acid in the sample will be absorbed by the silica surfaces (formed by the silica particles and alkyl silicate), which line the outer surfaces of the product and the insides of the fluid pathways.

The amount of nucleic acid adsorbed by the product is controlled by the surface area of silica available for binding to nucleic acid, which in turn is controlled by: the overall volume of the product, the ratio of the volume of polyethylene resin particles to the volumes of silica particles and alkyl silicate; and the sizes of silica particles (smaller particles have a larger surface area per unit volume). Controlling and limiting of the amount of nucleic acid captured by the product avoids the disadvantages associated with excess DNA/RNA for PCR amplification.

The proportion of magnetic beads will generally not affect nucleic acid adsorption because they are smaller in size and present in small amounts—and thus, have small volume in the product as compared with the resin particle and total silica volumes. The magnetic beads permit the product to be magnetically attracted allowing easier, or automated, movement.

The volumes of polyethylene resin particles silica particles in the product relate to the distribution of sizes of the particles. That is, the particle size ranges can be determined, and the numbers of each type of particles of a particular size controls the volume of that particle in the product. The numbers of each particle type can be averaged and standard distributions determined.

The product also allows extraction of DNA/RNA from limited volumes or numbers of cells. In the case of extraction from stem cells or cancer cell, few cells are generally available. The invention can efficiently extract DNA in sufficient quantities for PCR amplification, from as little as 5 μl of solution or as few as 1-100 cells.

The selection of polyethylene resin particles of a certain size range (e.g., 8-300 μm) also allows the product to function as a filter, allowing preferential selection of DNA material of certain sizes. For instance, one could select the smaller mitochondrial DNA by reducing the size of the resin particles so that the larger DNA cannot pass through the pathways, and only the smaller size DNA is captured.

It is an advantage to have several final products bearing the same patient nucleic acid, as they can be separately analyzed, or stored for later analysis. As DNA analysis for disease diagnosis and treatment, identification, genetic cross-matching and other purposes increases, a system for long term nucleic acid retention is increasingly useful. To identify several final products with one patient, the product can be bar-coded, or identified with another coded identification system (including RFID or nucleic acid tags), so that patient confidentiality can be maintained.

In preferred operation, the marked products are placed into a container and a patient's blood or fluid sample is added to the container, whereby nucleic acid in the blood or fluid sample is adsorbed by the silica surfaces. After adsorption, the products are removed from the container, preferably using a magnetic device, which attracts the magnetic beads to pull them from the container.

When marked/identified products are used, the patient's nucleic acid is not separated from the product, by elution or otherwise, before the nucleic acid is amplified. The marked products are placed in a container for amplification and the amplification is carried out on the solid phase product. The amplified nucleic acids in the container can be analyzed in situ, or the fluid in the container can be removed and they can be analyzed separately. The marked product is stable and can be stored for later use and analysis.

Because elution of nucleic acid from the product is eliminated as a step in the process, the process can also be readily automated. A robot simply uses magnetic attraction to lift the product from the sample container, and it is then placed in the container where PCR or other amplification, and analysis, takes place. Alternatively, an operator can perform this step.

### BRIEF DESCRIPTION OF THE DRAWINGS

FIG. 1 is plan view of the product for nucleic acid extraction and storage.

FIG. 2 schematically depicts the components of the product of FIG. 1 in a mold inside an oven.

FIG. 3 shows the work-flow for the capture of nucleic acid from a patient sample with the product of FIGS. 1 and 2.

FIG. 4 shows the real time PCR result where DNA was extracted from 100 cells. The 13 ng and 130 ng of DNA are controls, using such quantities of DNA.

FIG. 5A shows the work-flow for nucleic acid extraction from few cells.

FIG. 5B shows the extraction process from microliter volumes of a sample with high cellular concentration (like blood).

### DETAILED DESCRIPTION

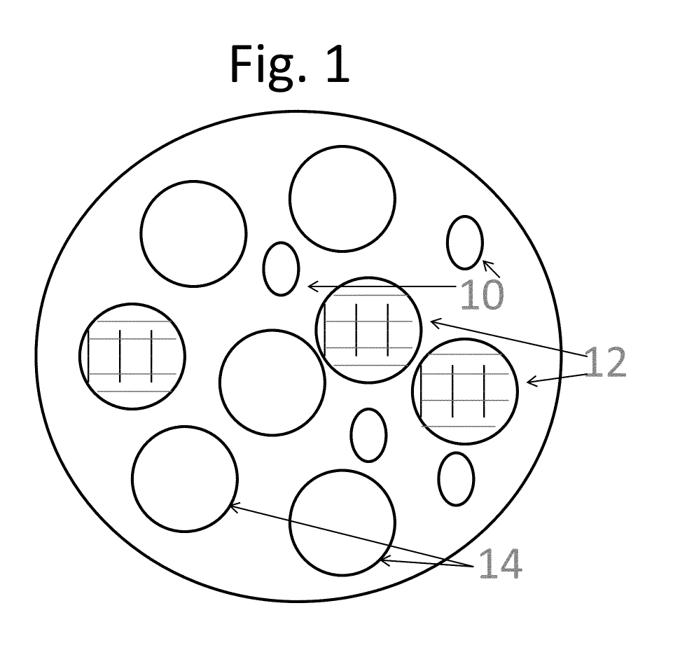

FIG. 1 is a plan view of the product for nucleic acid extraction. It includes magnetic beads 10, silica particles 12, alkyl silicate (not shown) and polyethylene resin particles (not shown) heated to weld the polyethylene resin particles together, thereby forming fluid channels (depicted as holes 14 in FIG. 1) through the product. Silica particles 12 are preferably about 5-20 μm in diameter. Polyethylene resin particles range from 8-1000 μm in diameter. Magnetic beads can be smaller, preferably from 0.1 to 2 μm in diameter. Controlling the relative proportion and size range of the polyethylene resin particles used in the product controls the size, the distribution of sizes and the number of fluid channels in the product, and the quantity of nucleic acid it can adsorb and ultimately release.

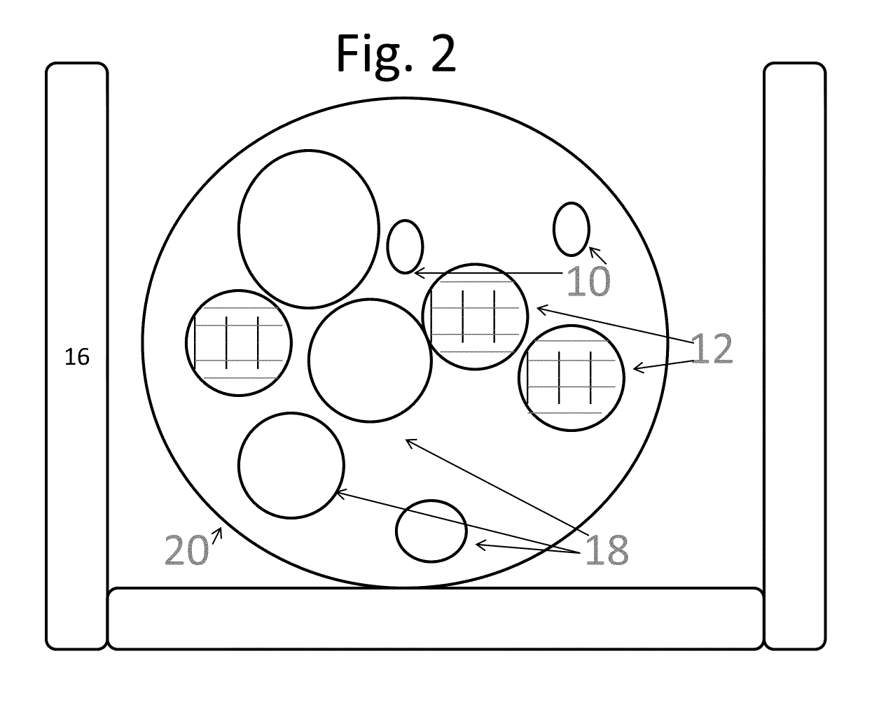

FIG. 2 depicts making the product, i.e., mix the ingredients (silica particles 12, alkyl silicate, not shown, and resin particles 18) in a mold 20 to form a three-dimensional structure (a disc in this case, but other shapes can be used), and place the mold 20 inside oven 16. Preferably, the oven is heated to 200° C., cooled to room temperature and release from the mold, then heated to 500° C. for one hour to finish the particle welding process.

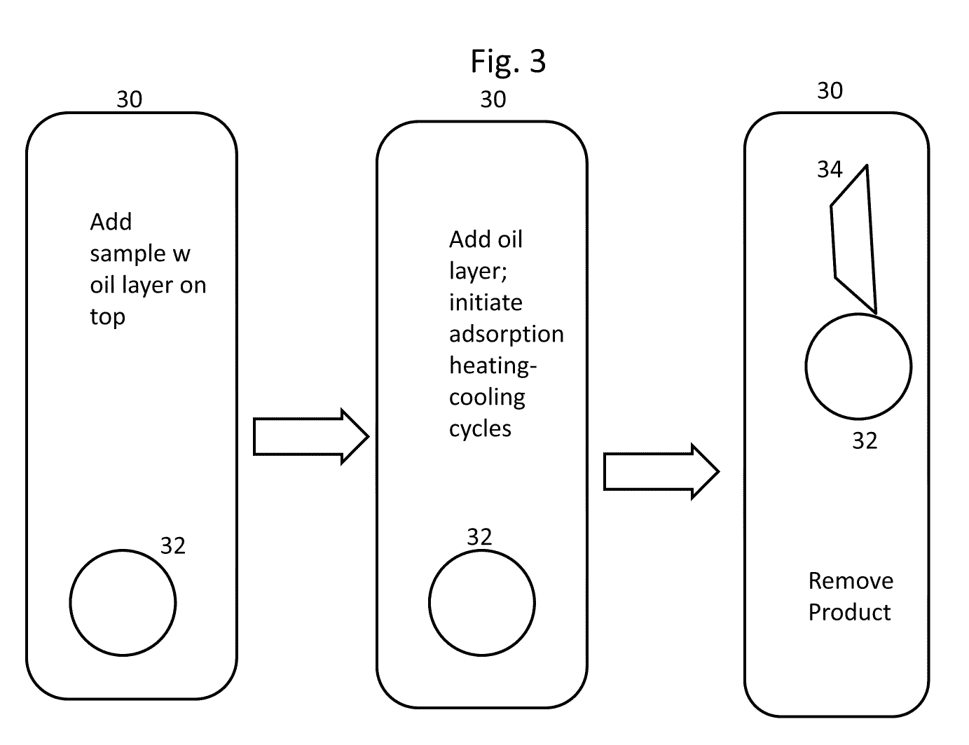

FIG. 3 depicts adsorbing DNA with a product 32 in the lower part of a tube 30, which is preferably coded using, e.g., a bar code, to identify the patient source. A sample (blood or another patient sample, or a sample from plants, that contains DNA or RNA) with an oil layer on top, is transferred to the tube 30. Tube 30 can contain one or more of the products 32, which are preferably encoded e.g., with a bar code. Alternatively, the encoding can be by nucleic acid tagging or by comparing the unique patterns on each product (which are formed in its making). These patterns are stored as images which can be decoded later by taking another image and comparing, in order to identify any particular product.

The tube 30 also contains all the reagents needed for adsorption of DNA or RNA from the sample. Further oil is added on the upper surface of the sample, to protect the sample from airborne particles and contamination, and to isolate the sample (potentially a biohazard) from the work area and inhibit evaporation.

The sample is then put through a heating and cooling cycle—a typical cycle would be room temperature to 45° C. to 85° C. for 1-10 min, then RT-60° C. for 1-10 min to adhere nucleic acid to the product. The heating/cooling cycle runs preferentially at 65-81° C., then between RT and 48° C. for 5-20 cycles. Once the cycle finish, washing reagents (typically at 1-1000 μl) would be added to the tube to dilute the sample. Or the sample can be taken to a washing station for washing

A magnetic device 34 can be used to pick the product out of the tube (by attraction to the magnetic beads) and transfer it to a washing station for more extensive washing. Picking with device 34 can be part of an automated system—a robot can be controlling it, and initiating this action at the appropriate time. Also, the robot could place the product in tube 30 initially, then transfer at the appropriate time.

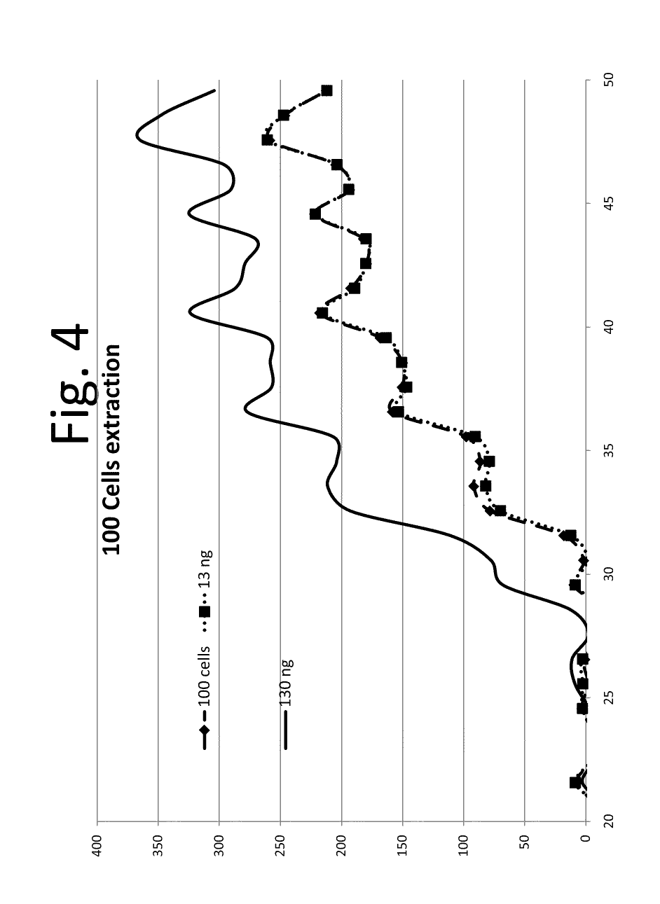

FIG. 4 shows the results of real time PCR where DNA was extracted with a product as described herein from 100 human cells. 13 ng of DNA was extracted from the cells. As controls, a sample of 13 ng and a sample of 130 ng were also ran on the real time PCR. The results are as shown. It can be seen that the 13 ng of DNA extracted from the cells appears to follow the same amplification pattern as the control.

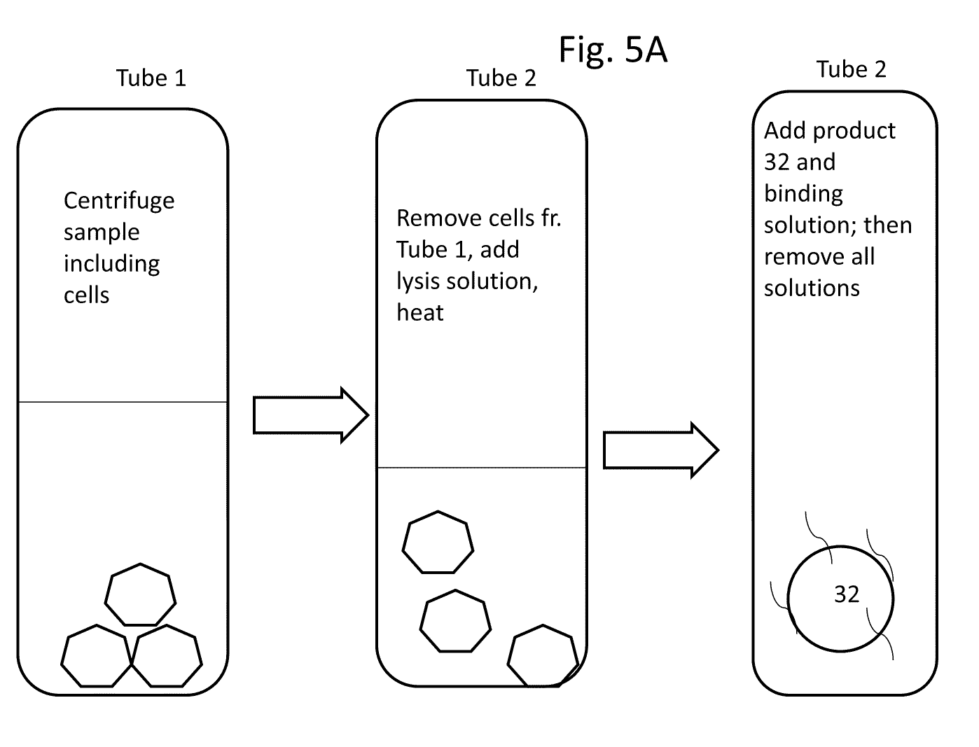

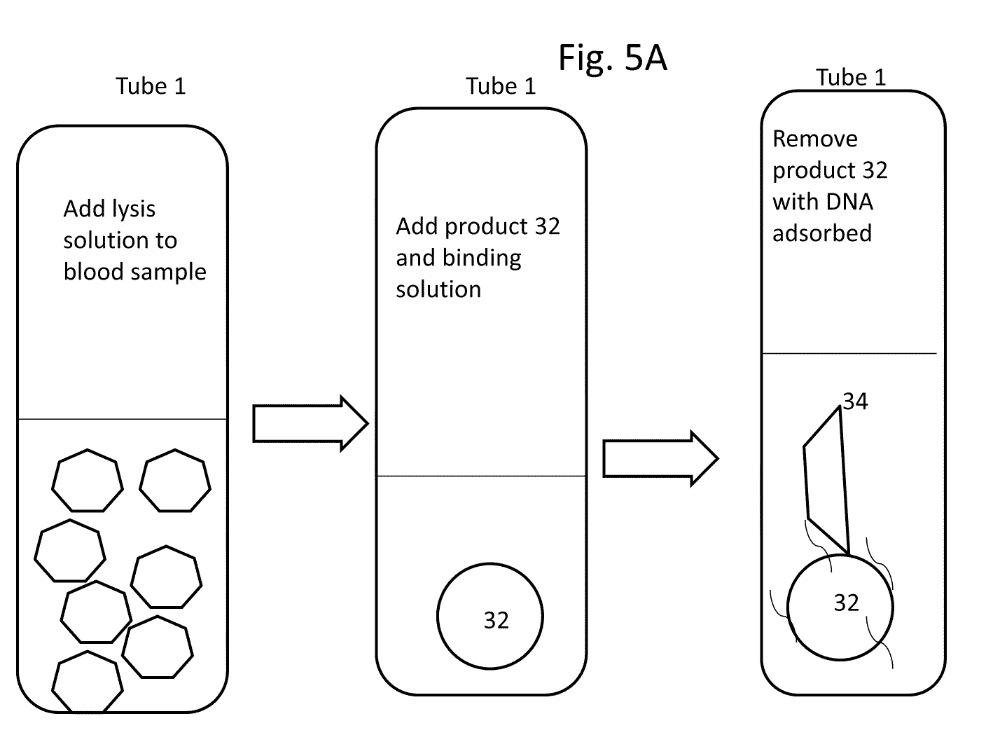

FIG. 5A shows the extraction process from very few cells. First, one centrifuges the input sample—which can be as little as 5 to 200 μl. After spinning the cells at about 14,000 rpm for 10 minutes, the cells accumulate at the bottom of the tube. Excess solution is removed leaving only the cells. Next, 5 to 200 μl of a lysis solution (1:1 ratio) is added, which consists of:

10 ul GuTE; 5 M Guanidine HCl; 50 mM Tris HCl; 10 mM EDTA (Ethylenediaminetetraacetic acid) in dd water; and 1 ul Proteinase K
and the solution is incubated at 75° C. for 10 minutes. Next, two products 32 (each with dimensions 1×1×2 mm) are added at a 1:1 ratio to a mixture of the lysis solution with 5-200 μl of a binding solution (which is 100% ethanol), followed by incubation at 75° C. for 10 minutes. All solutions are then removed. The product 32 will have captured DNA from the cells.

FIG. 5B show the extraction process for a low volume of a solution that contains a high concentration of cells—like blood. There is no need to centrifuge and concentrate the cells before adding the lysis solution—it is added directly.

To determine the maximum amount a product as described herein can capture, 20 μl of a 1000 ng/ul DNA solution is used. The exemplary product for extraction is 1×1×1.5 mm and is incubated for 5 minutes after sample contact, and eluted with 10 μl water. As shown in Table I, by changing the proportion of resin in the product (where the resin particles are a particular size range and size range distribution) the amount of DNA released varies in accordance with the change in adsorbing surface area of the product. The amount of DNA adsorbed and released by the product will change with changes in the proportion of resin and the size range and size range distribution of the polyethylene resin particles, as explained above. In Table 1, the particle size ranges from 8-1000 μm in diameter, as determined by observation of a population of the particles. Populations of particles with size ranges of 8-1000 μm in diameter (assuming a similar size distribution in that range) when used in the same proportions as in Table I in making the product, would form products which would be expected to release the same amounts of DNA as indicated in Table I. If the distribution of sizes of the polyethylene resin particles shifts towards larger particles, the distribution of the fluid channel diameters will also increase, and the product will release more DNA. Similarly, shifting the distribution of particle sizes to smaller particles will result in a decrease in the amount of DNA the product releases.

Table 1 shows the experimental results where the proportion of polyethylene resin particles in the product (by volume) increases from 0 to 35% (particle size varies from 8-1000 μm). The product captures from 200 ng of DNA (where no resin was in product), 300 ng at 15%, 400 ng at 25% and 500 ng of DNA where resin is 35% by volume of the product. The experimental conditions were: for each run of product with a different proportion of resin: 10 μl of 1000 ng/μl pure DNA was contacted with two products as described herein (each was 1×1×1.5 mm, and with the proportion of resin and particle sizes indicated in Table I) in binding solution. This was followed by incubation at 75° C. for 5 minutes, and elution at room temperature with water for 5 minutes.

TABLE I
ng of DNA captured in right hand column

| Resin % | |
|---|---|
| by volume | |
| 0 | 200 |
| 15 | 300 |
| 25 | 400 |
| 35 | 500 |

The ability to adjust the amount of DNA released is highly desirable for downstream PCR. The product described herein allows the amount of DNA released to be adjusted for optimal PCR.

It should be understood that the terms and expressions used herein are exemplary only and not limiting, and that the scope of the invention is defined only in the claims which follow.

What is claimed:

1. A process of making a product for capture of nucleic acid from a sample, comprising:
heating a mixture of magnetic beads, silica particles, alkyl silicate and polyethylene resin particles so as to weld the polyethylene resin particles together, and such that fluid pathways are formed through the product; and
controlling the surface area of silica available for binding nucleic acid by controlling: (i) the overall volume of the product; (ii) the ratio of the volume of polyethylene resin particles to the volumes of silica particles and alkyl silicate; and (iii) the sizes of silica particles, so as to control the amount of nucleic acid captured and ultimately released from a product.

2. The process of claim 1 wherein the proportion of polyethylene resin particles in the product varies from 0 to 35% by volume.

3. The process of claim 1 wherein the size range of the polyethylene resin particles is from 8-1000 μm in diameter.

4. The process of claim 1 wherein the size range of the polyethylene resin particles is from 8-200 μm in diameter.

5. The process of claim 1 wherein the samples are patient blood, tissues, feces or fluid samples.

6. A process of claim 1 wherein the samples are 100 or fewer patient cells.

7. The process of claim 1 wherein the alkyl silicate includes ethyl polysilicates.

8. The process of claim 1 wherein the nucleic acid is DNA or RNA.

9. The process of claim 1 further including the step of determining the distribution of the size range of polyethylene resin particles or silica particles.

10. The process of claim 9 wherein the distribution is defined as a specified proportion of the particles being within a specified size range or of a specified size.

11. The process of claim 1 wherein the standard deviation of the particles within the size range is determined.

12. A process of using the product generated by the process of any of claims 1 to 11 for capture and analysis of nucleic acid from a sample.
